(19) United States
(12) Patent Application Publication
(10) Pub. No.: US 2021/0046228 A1
(43) Pub. Date: Feb. 18, 2021

(54) Arrangement with a Blood Pump and Pump Control Unit

(71) Applicant: Xenios AG, Heilbronn (DE)

(72) Inventors: Holger Gorhan, Schoenaich (DE); Georg Matheis, Heilbronn (DE)

(21) Appl. No.: 17/086,965

(22) Filed: Nov. 2, 2020

Related U.S. Application Data
(63) Continuation of application No. 14/444,248, filed on Jul. 28, 2014.

(30) Foreign Application Priority Data
Jul. 29, 2013 (DE) 102013012433.6

(51) Int. Cl. A61M 1/10; A61M 1/26; A61M 1/16

(52) U.S. Cl. CPC A61M 1/1006; A61M 1/267; A61M 1/1698; A61M 1/1086; A61M 2230/30; A61M 2205/3334; A61M 1/101; A61M 2205/3341; A61M 2230/04; A61M 1/1005

(57) ABSTRACT

An arrangement for extracorporeal life support is further developed in such a way that a pump actuating signal produces a wave-like surging and subsiding pump output for a pulsatile flow. The pump is preferably a non-occlusive blood pump, such as a diagonal pump, for example. In a preferred variant of embodiment the control signal is provided by an ECG. This allows the diastolic pressure to be increased in order to improve the oxygen balance of the heart muscle.

### CROSS-REFERENCE TO RELATED APPLICATIONS

This application is a continuation application of and claims priority to U.S. application Ser. No. 14/444,248, filed on Jul. 28, 2014, which claims priority under 35 U.S.C. § 119 of German Application No. 10 2013 012 433.6 filed on Jul. 29, 2013, the disclosures of which are expressly incorporated herein in its entirety by reference thereto.

### TECHNICAL FIELD

The blood pump relates to an arrangement with a blood pump and a pump control unit which has a computer that converts a control signal into a pump actuating signal.

### BACKGROUND

Such arrangements are used for extracorporeal life support (ECLS) for example.

ECLS is used, for example, in patients with cardiogenic shock or decompensated heart failure, whose heart is no longer able to supply the body sufficiently with oxygen-rich blood.

### SUMMARY

The purpose of the invention is to further develop such an arrangement and to propose a method of operating a blood pump.

This objective is achieved with an arrangement of the type in question in which the pump actuating signal brings about a wave-like surging and subsiding pump output for a pulsatile flow. The pulsatile flow produced by the pump actuating signal improves the circulatory situation.

A wave-like surging and subsiding pump output does not mean a constant pump stroke or switching the pump on and off, but a pump output that is produced by a variable control signal and varies over time.

The arrangement makes a cardiac support system possible that emits pulses integrated into the cardiac cycle in order to improve the blood supply to the coronary vessels and better supply the heart with oxygen.

It is advantageous if the blood pump also provides a constant basic output. In this way the systemic perfusion pressure is increased with a laminar base flow.

This constant basic output can be provided by the pump which also brings about the pulsatile flow. Depending on the area of application it may be advantageous for the arrangement to have a further blood pump which provides the constant basic output.

In this case the further pump can also provide a wave-like surging and subsiding pump output.

In this way the pulsatile flow and the constant basic output can be provided either by means of one pump or the surging and subsiding pump output and constant basic output functions are split between two pumps.

However, two pumps can also be used which each provide a wave-like surging and subsiding pump output. With a second pump time operating in a time-delayed manner with regard to the first blood pump, it is possible to provide a wave-like surging and subsiding pump output so that the pressures waves overlap.

Such an arrangement usually has an oxygenator which is supplied by the pump. In principle the pump can be arranged either upstream or downstream of the oxygenator. It is of advantage if one blood pump is arranged upstream of the oxygenator in the direction of flow and a further blood pump is arranged downstream of the oxygenator.

A preferred variant of embodiment envisages that the oxygenator has a housing and that at least one blood pump is arranged in this housing. This makes it possible to arrange, for example, a blood pump in the housing of the oxygenators upstream of the oxygenator or downstream of the oxygenator.

A particularly advantageous variant of embodiment envisages that the arrangement has at least one non-occlusive blood pump, such as, in particular, a diagonal, axial or centrifugal pump.

In order to provide the required control signal it is envisaged that the arrangement has a clock generator. In accordance with a predetermined rhythm, this clock generator can provide the control signal for the pump in terms of frequency and amplitude. In this way the wave-like surging and subsiding pump output is achieved.

In a particularly preferred variant of embodiment this control signal is provided by an ECG. For this, software with the ability to record an ECG signal is integrated into the control unit of an ECLS system. A patient cable derives the ECG signal on the patient. Preferably the thus recorded R wave is the clock generator (trigger) for emitting a software trigger for starting the blood pump which then generates the pulse. The software ensures the precise emission of the pulse to the cardiac cycle, preferably the diastole. Advantageously it is ensured that the duration of the pulse is adapted in such a way that at the start of systole the pulse is no longer present. However, a pulse profile can also be generated which acts on the systole and/or on the diastole.

Cumulatively or alternatively it is proposed that the arrangement has an arterial pressure sensor which provides the control signal. This makes it possible to influence the pump output by means of a pressure measurement on an artery.

Experience has shown that it is advantageous if the arrangement has an arterial cannula which is longer than around 20 cm, preferably longer than 30 cm. The particularly long cannula serves to ensure that the pulse is emitted as closely to the heart as physiologically possible.

The aim on this the invention is based is also achieved with a method for operating a blood pump, in which the pump is operated with an iterating output in order to produce a wave-like surging and subsiding pulsatile flow.

Phase-shifted in relation to the pulsatile flow, a further blood pump can bring about a wave-like surging and subsiding pump output.

It is advantageous if the pulsatile flow of at least one pump is overlapped by a base load.

In the implementation of the procedure it is preferably ensured that the diastolic pressure is increased with the pump. This allows the circulation support to be produced with an ECLS system in such a way that in addition to a laminar base flow the pulsatile function is adjusted so that a flow and pressure increase takes places in the diastole phase. Triggering of the system preferably takes place through synchronisation with the heart.

The described arrangement can, however, also be used to direct the flow to an oxygenator with the pump. The pulsatility improves the function and service life of the oxygenator.

### BRIEF DESCRIPTION OF THE DRAWINGS

FIG. 1 illustrates an arrangement for extracorporeal life support.

FIG. 2 illustrates another arrangement for extracorporeal life support.

### DETAILED DESCRIPTION

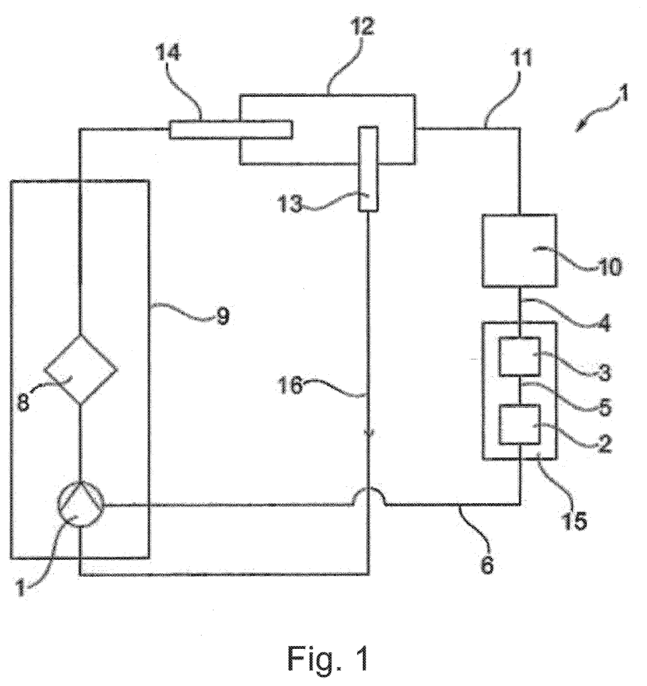

Essential elements of the arrangement 1 are a first blood pump 1, a pump control unit 2 and a computer 3, as shown in FIG. 1. The computer 3 converts a control signal 4 into a pump actuating signal. Via the pump control unit 2 this pump actuating signal 5 produces a wave-like surging and subsiding pump output on the pump 1 which thereby brings about a pulsatile flow.

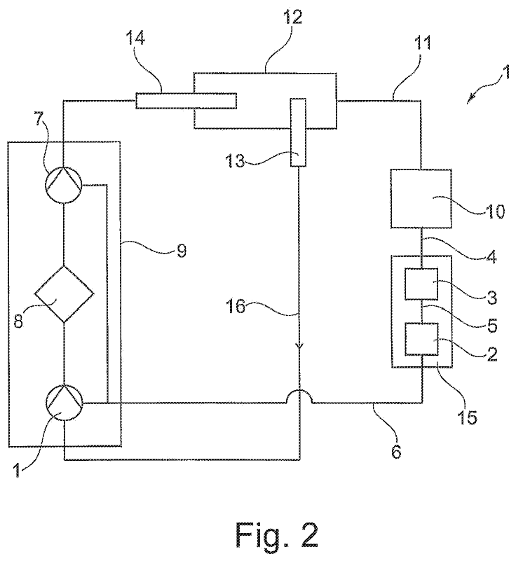

Via the lead 6, the pump control unit 2 is connected to the first pump 1 and a further pump 7, as shown in FIG. 2. This makes it possible to produce both basic load and also pulsatile flow with the first pump 1 which is arranged upstream of an oxygenator 8. However, with the first pump 1 upstream of the oxygenator 8 a basic load can also be produced, and with the second pump 7 downstream of the oxygenator 8 a pulsatile flow.

Finally, in each case a pulsatile flow can also be achieved with the first pump 1 upstream of the oxygenator 8 and the second pump 7 downstream of the oxygenator. Because of the distance between the pumps, this makes it possible to overlap time-delayed waves or to control the pumps with time delayed signals.

Together with the oxygenator 8, the pumps 1 and 7 are arranged in a housing 9. This permits a simple construction. In the shown example of embodiment only one lead 6 runs from the pump control unit 2 to the housing 9 in order in the housing 9 to provide the two pumps 1 and 7 with a pump actuating signal. As an alternative one lead can be taken to the first pump 1 and a further lead to the second pump 7.

As a blood pump a diagonal pump is used, at least for the first pump 1. Preferably both pumps 1 and 7 are diagonal pumps. However, axial or centrifugal pumps can also be used.

The control signal 4 is provided by an ECG 10 which is connected to the patient 12 via a cable 11.

Located in the blood circulation or heart of the patient 12 are a venous cannula 13 and an arterial cannula 14. The arterial cannula is around 35-40 cm, preferably 30 to 45 cm, in length and the venous cannula is introduced into the vena cava.

During operation of the ECLS system, with the ECG 10, via the lead 11 an ECG signal of a patient 12 is recorded in order to generate a control signal 4. This control signal 4 is converted by the computer 3 into a pump signal 5 which, via the pump control unit 2 and lead 6 controls the pumps 1 and 7 or provides them with a current. A console 15 is used which emits a software trigger to start the blood pump 1 in accordance with a specially developed algorithm with the aim of emitting impulses into the systole and/or the diastole.

For this the ECG signal is implemented in the console. The user interface is adapted in order to create settings options for the ECG and to constitute a marker channel to show the relevant action of the blood pump as a sense or pulse.

In the blood circulation 16 from the venous cannula 13 to the arterial cannula 14 the blood is enriched with oxygen in the oxygenator 8 and CO₂ is removed.

The blood pump 1 is accelerated by a special value on top of the base speed for a defined period within a maximum time window which is dependent on the current heart frequency. The time limitation takes place by way of a further algorithm.

The blood pump or blood pump 1 and 7 are controlled in such a way that a diastolic augmentation occurs. During this heart action the coronary perfusion pressure is increased. The end-diastolic blood pressure in the area of the aorta close to the heart then falls to a lower value than normal. The following systole has less ejection resistance to overcome and is therefore known as an “influenced systole”. The lower afterload can be seen in the lower systolic pressure.

By increasing the diastolic pressure the oxygen balance of the heart muscle is improved in two ways: the myocardial oxygen supply is increased by a rise in the coronary perfusion pressure and at the same time the mechanical heart action and thereby the myocardial oxygen consumption are decreased. In this way the preconditions for recovery of the heart are improved.

One problem of oxygenators is clotting, whereby the constituents of the blood are deposited on the gas exchange membrane. In addition, clots can form in areas of the oxygenator where there is little flow. Through the pulsatile flow through the oxygenator the flow in the oxygenator changes, as a result of which the service life of the oxygenator is improved.

Furthermore, as a side effect the gas exchange is improved as the boundary layer between fibres and the flowing blood is reduced.

1. (canceled)

2. An extracorporeal life support system comprising:
a blood line set configured to be connected to a patient for receiving blood from the patient and returning the blood to the patient;
a single blood pump connected to the blood line set and configured to pump the blood through the blood line set;
an ECG device for measuring a cardiac cycle of the patient; and
a pump control unit configured to be connected to (i) the ECG device for receiving a control signal from the ECG device and (ii) the single blood pump for transmitting a pump actuating signal to the single blood pump,
wherein the pump actuating signal is configured to cause the single blood pump to generate a pulsatile blood flow that overlaps with a base blood flow, and the pump actuating signal is configured, based on the control signal received from the ECG device, to cause the single blood pump to generate the pulsatile blood flow in a manner such that the pulsatile blood flow is present during a diastole phase of the cardiac cycle of the patient and is no longer present at a start of a subsequent systole phase of the cardiac cycle of the patient.

3. The extracorporeal life support system of claim 2, wherein the pump actuating signal is configured to operate the single blood pump at an increased speed to generate the pulsatile blood flow during the diastole phase of the cardiac cycle of the patient.

4. The extracorporeal life support system of claim 2, wherein the control signal is a variable control signal that varies over time.

5. The extracorporeal life support system of claim 2, wherein the base flow is a laminar base flow.

6. The extracorporeal life support system of claim 2, wherein the single blood pump is a non-occlusive blood pump.

7. The extracorporeal life support system of claim 2, wherein the single blood pump is a diagonal blood pump.

8. The extracorporeal life support system of claim 2, further comprising an oxygenator, the single blood pump being configured to pump the blood to the oxygenator.

9. The extracorporeal life support system of claim 2, wherein the pump control unit is configured to record the control signal received from the ECG device.

10. The extracorporeal life support system of claim 2, wherein the pump control unit comprises a computer configured to convert the control signal into the pump actuating signal.

11. The extracorporeal life support system of claim 2, further comprising an arterial pressure sensor.

12. The extracorporeal life support system of claim 2, wherein the blood line set comprise an arterial cannula for receiving the blood from the patient, the arterial cannula having a length greater than 20 cm.

13. The extracorporeal life support system of claim 12, wherein the arterial cannula has a length greater than 30 cm.

14. The extracorporeal life support system of claim 12, wherein the arterial cannula has a length of 30-35 cm.

15. The extracorporeal life support system of claim 12, wherein the arterial cannula has a length of 35-40 cm.

16. The extracorporeal life support system of claim 12, wherein the blood line set further comprises a venous cannula.

17. The extracorporeal life support system of claim 2, wherein the pump actuating signal is configured to cause the single blood pump to generate the pulsatile blood flow within a time window that is dependent on a heart rate of the patient.

18. The extracorporeal life support system of claim 2, wherein the pump actuating signal is configured to cause the single blood pump to operate at accelerated speed for a defined period within a maximum time window which is dependent on a current heart rate of the patient.

19. The extracorporeal life support system of claim 2, wherein the pump actuating signal is configured to ensure precise emission of the pulsatile flow in the diastole phase of the cardiac cycle of the patient.

20. The extracorporeal life support system of claim 2, wherein the control signal received from the ECG device is provided by a clock generator in accordance with a predetermined rhythm.

21. The extracorporeal life support system of claim 20, wherein the clock generator is a recorded R wave recorded by the ECG device.

22. An extracorporeal life support method comprising:
receiving, by a pump control unit, a control signal from an ECG device connected to a patient; and
transmitting, by the pump control unit, a pump actuating signal to a single blood pump connected to a blood line set connected to the patient to generate a pulsatile blood flow that overlaps with a base blood flow, the pump actuating signal being based on the control signal received from the ECG device, the pulsatile blood flow being present during a diastole phase of a cardiac cycle of the patient and no longer present at a start of a subsequent systole phase of the cardiac cycle of the patient.
